(19) United States
(12) Patent Application Publication
(10) Pub. No.: US 2024/0148684 A1
(43) Pub. Date: May 9, 2024

(54) COMPOUNDS AND METHODS FOR TREATING CONGENITAL ERYTHROPOIETIC PORPHYRIA

(71) Applicants: RUTGERS, THE STATE UNIVERSITY OF NEW JERSEY, New Brunswick, NJ (US); REGENTS OF THE UNIVERSITY OF MICHIGAN, Ann Arbor, MI (US)

(72) Inventors: Bishr OMARY, New Brunswick, NJ (US); Jordan Adam SHAVIT, Ann Arbor, MI (US)

(73) Assignees: RUTGERS, THE STATE UNIVERSITY OF NEW JERSEY, New Brunswick, NJ (US); REGENTS OF THE UNIVERSITY OF MICHIGAN, Ann Arbor, NJ (US)

(21) Appl. No.: 18/278,904

(22) PCT Filed: Feb. 24, 2022
(86) PCT No.: PCT/US2022/017743

§ 371 Date: Aug. 25, 2023

Related U.S. Application Data
(60) Provisional application No. 63/154,292, filed on Feb. 26, 2021.

(51) Int. Cl. A61K 31/203; A61K 9/00; A61K 31/409; A61P 1/16

(52) U.S. Cl. CPC A61K 31/203; A61K 9/0019; A61K 31/409; A61P 1/16

(57) ABSTRACT

The invention provides a method for treating or ameliorating the clinical manifestations of congenital erythropoietic porphyria in an animal (e.g. a human) by administering a retinoid to the animal.

### PRIORITY

This application claims priority from U.S. Provisional Patent Application No. 63/154,292, which was filed on 26 Feb. 2021. The entire content of this United States Provisional Patent Application is hereby incorporated by reference herein.

### GOVERNMENT FUNDING

This invention was made with government support under DK116548 awarded by the National Institutes of Health. The government has certain rights in the invention.

### BACKGROUND

Congenital erythropoietic porphyria (CEP) is a rare genetic disorder leading to accumulation of uro/coproporphyrin-I in tissues due to inhibition of the enzyme uroporphyrinogen-III synthase. Clinical manifestations of CEP include bone fragility, severe photosensitivity and photo-mutilation. Currently there is no specific treatment for CEP, except bone marrow transplantation. There is a clear need for safe and effective agents for treating congenital erythropoietic porphyria.

### SUMMARY

In one aspect the present invention provides a method for treating congenital erythropoietic porphyria in an experimental animal model, comprising administering a retinoid or a pharmaceutically acceptable salt thereof to the animal.

The invention also provides a pharmaceutical composition for treating congenital erythropoietic porphyria comprising a retinoid or a pharmaceutically acceptable salt or carrier thereof, and a pharmaceutically acceptable excipient.

The invention also provides a retinoid or a pharmaceutically acceptable salt or carrier thereof for the prophylactic or therapeutic treatment of congenital erythropoietic porphyria.

The invention also provides the use of a retinoid or a pharmaceutically acceptable salt or carrier thereof to prepare a medicament for treating for treating congenital erythropoietic porphyria in an animal (e.g. a mammal such as a human).

The invention also provides a method comprising:
injecting a porphyrin (e.g. Uro-I) into a zebrafish;
contacting the zebrafish with a target compound in a medium;
measuring the accumulation of the porphyrin in the bones or other tissue of the zebrafish;
comparing the accumulation of the porphyrin in the bones or other tissue of the zebrafish with a control to determine whether the target compound reduced porphyrin accumulation in the bones or other tissue of the zebrafish; and
optionally determining the amount of porphyrin in the medium.

### BRIEF DESCRIPTION OF THE FIGURES

FIGS. 1A-1E. Zebrafish model of CEP develops bone phenotype resembling human disease: (FIG. 1A) 6 dpf zebrafish larvae were injected with uro-I or vehicle and imaged by confocal microscopy at 7 dpf. Porphyrin was detected only in the bones of uro-I-injected group. Arrowhead corresponds to the operculum; box corresponds to vertebrae. (FIG. 1B) Larvae were treated as in (A) and injected with calcein prior to imaging. Arrowhead (operculum); arrow (4^{th} vertebra). (FIG. 1C) Quantification of bone volume in larvae from (B); bone volume was normalized to vehicle-injected larvae set to 100%. Symbols represent individual larvae (14-18/group) from 4-5 independent experiments. (FIG. 1D) Larvae were treated as in (A). At 7 dpf, bones were harvested and imaged by epifluorescence microscopy pre- and post-HCl bone demineralization; arrow (notochord). (FIG. 1E) Hydroxyapatite was incubated with calcein/uro-I/copro-I and imaged by epifluorescence microscopy. Scale bars: 200 μm (A-D), 50 μm (E); asterisks highlight statistical significance between the indicated groups.

FIGS. 2A-2J. Acitretin mitigates CEP bone phenotype in zebrafish: (FIG. 2A) 6 dpf larvae were injected with uro-I and transferred to medium containing acitretin or DMSO. At 7 dpf, larvae were injected with calcein and imaged by confocal microscopy. Quantification of porphyrin fluorescence (FIG. 2B), operculum volume (FIG. 2C) and porphyrin excretion (FIG. 2D) from experiment in (A). Symbols represent individual larvae (12-25/group) from 3-4 independent experiments. (FIG. 2E) 6 dpf larvae were injected with uro-I. At 7 dpf they were transferred to medium containing acitretin or DMSO. At 8 dpf larvae were injected with calcein and imaged by confocal microscopy. Quantification of porphyrin fluorescence (FIG. 2F), operculum volume (FIG. 2G) and porphyrin excretion (FIG. 2H) from experiment in (E). Arrowhead, operculum; box, vertebrae (A, E). Symbols represent individual larvae (18-64/group) from 3-4 independent experiments. (FIGS. 2I, J) Larvae were treated as in (A) with the indicated retinoid or DMSO and porphyrin fluorescence (I) and operculum volume (J) were assayed. Bone volume was normalized to DMSO-treated larvae set to 100%, (C, G, I). Symbols represent individual larvae (7-17/group) from 2 independent experiments. Scale bars: 200 μm; asterisks highlight statistical significance between the indicated groups.

FIGS. 3A-3I. Saos-2 cells mimic CEP zebrafish model: (FIGS. 3A, B) Mineralization in Saos-2 cells treated with MAC±Uro-I was assayed using ARS staining (photograph, A; quantification, B). Staining was normalized to MAC only-treated cells (set to 100%). (FIG. 3C) Cell lysates from experiment in (A) were blotted with antibodies to the indicated antigens. (FIG. 3D) Quantification of LC3-II shown in (C). LC3-II level was normalized to MAC only (left panel) or vehicle-treated (right panel), set to 100%. (FIG. 3E) RT² Profiler PCR Array (left panel) and qPCR (right panel). Relative gene expression is represented as fold-change normalized to housekeeping gene. Data are from 2 independent experiments. (FIG. 3F) Acitretin does not rescue reduced mineral matrix phenotype in uro-I-treated cells. ARS staining quantification as in (B). (FIG. 3G) Acitretin normalizes ER stress (BiP) and autophagy (LC3-II) markers. Dashed lines represent non-adjacent lanes in the gel. Coomassie-stained gel (C, G) shows equal protein loading. (FIG. 3H) Quantification of LC3-II. LC3-II level was normalized to DMSO-treated cells set to 100%. (FIG. 3I) Gene expression profiling as in (E). Asterisks highlight statistical significance between the indicated groups.

FIG. 4. Proposed model of CEP pathogenesis: UROS inhibition leads to production of uro/copro-I mostly in erythrocytes and liver, which is transported through blood to the bones. Uro-I causes bone damage by binding to hydroxyapatite, causing oxidative and ER stress, protein aggregation and stalled autophagy. Acitretin partially rescues uro-I-induced bone damage by reducing oxidative and ER stress and restoring autophagic flux.

FIG. 5. UROS inhibition accumulates uro-I and copro-I in CEP. UROS, a cytosolic enzyme, catalyzes the conversion of the linear tetrapyrrole, hydromethylbilane (HMB) to the first cyclic tetrapyrrole of the pathway, uroporphyrinogen-III (Ajioka, R. S., et al., 2006, Biochimica et Biophysica Acta (BBA)—Molecular Cell Research, 1763, 723-736; and Layer, G., et al., 2010, Protein Sci, 19, 1137-61). UROS ‘flips’ the position of the acetate and propionate in the ‘D’ pyrrole ring and subsequently causes ring closure to form uroporphyrinogen-III (dotted oval) (Ajioka, R. S., et al., 2006, Biochimica et Biophysica Acta (BBA)—Molecular Cell Research, 1763, 723-736; and Phillips, J. D., et al., 2003, EMBO J 22, 6225-33). Uroporphyrinogen-III is decarboxylated by uroporphyrinogen decarboxylase (UROD) to form coproporphyrinogen-III, which through a multi-step mechanism that involves the formation of protoporphyrin-IX, generates heme. In absence of UROS activity, there is spontaneous ring closure of HMB to form uroporphyrinogen-I, a positional isomer of uroporphyrinogen-III, where the acetate/propionate inversion in ring ‘D’ does not occur. Uroporphyrinogen-I is decarboxylated by UROD to coproporphyrinogen-I, but after this step the pathway gets blocked since coproporphyrinogen-I cannot be metabolized by coproporphyrinogen oxidase. Porphyrinogens are relatively unstable compounds, and are auto-oxidized from their colorless, non-fluorescent porphyrinogen forms to colored, fluorescent porphyrins (Badminton, M. N. and Elder, G. H. (2014). CHAPTER 28—Clinical Biochemistry: Metabolic and Clinical Aspects (Third Edition), (eds W. J. Marshall M. Lapsley A. P. Day and R. M. Ayling), pp. 533-549: Churchill Livingstone). Thus UROS blockade leads to accumulation of uroporphyrin-I (uro-I) and coproporphyrin-I (copro-I).

FIG. 6. High throughput drug screening for CEP. High throughput drug screening protocol to identify potential drug treatments for CEP was conducted by testing 1,280 small molecules from the commercially available Prestwick library. Initial screening was performed by pooling four drugs per well, with two zebrafish larvae in each well. 6 dpf zebrafish larvae were injected with uro-I and calcein simultaneously. 24 h later, they were imaged by epiflourescence microscopy using the automated ImageXpress system. Visual analysis was conducted and identification of wells containing larvae with reduced uro-I and increased calcein signal (magenta and green arrows, respectively) in bones compared to DMSO-treated larvae were selected for individual testing of each drug. Of the 320 pools tested, one was identified as potential hit. Once the four drugs were tested individually, acitretin was identified for decreasing uro-I accumulation in bones.

FIGS. 7A-7C. Uro-I causes aggregation of bone matrix proteins in a light-independent manner. (FIG. 7A) Saos-2 cells were treated for three days with uro-I or vehicle in the presence of mineralization activation cocktail (MAC). Cells grown in medium without MAC (no mineralization stimuli) and in MAC alone were used as controls for MAC efficiency. Experiments were performed in a dark room and cells were shielded from light throughout the whole experiment. In order to verify whether protein aggregation took place while cells were alive and represented a biologically relevant finding, or if aggregation was an artifact of light exposure during processing of samples, an aliquot of lysate from uro-I treated cells was exposed to light prior to addition of reducing SDS-PAGE sample buffer, which we have shown previously that prevents light-induced protein aggregation by porphyrins in cell lysate. Uro-I treatment did not cause lamin A/C to aggregate, with monomer being comparable between vehicle- and uro-I treated cells (3^{rd} and 4^{th} lanes, short exposure). However, upon light exposure of the uro-I treated cells lysate, loss of monomer and high molecular aggregates were observed (7^{th} and 8^{th} lanes, long exposure). These findings confirm that accidental light exposure of samples did not happen, and any protein aggregation observed was a true biological event, not an artifact of cell processing. (FIG. 7B) We conducted a proteomics experiment of cell lysates treated with uro-I and vehicle in the dark to further confirm our findings that bone matrix proteins aggregated upon uro-I treatment. Six (1 cm) regions of a Coomassie stained gel (I-IV, cartoon) spanning from the bottom of the well to slightly above the 75 kDa marker were cut and submitted to mass spectrometry analysis. (FIG. 7C) Our results confirmed lamin A/C aggregated only in the light-exposed uro-I treated cells lysate, but not in vehicle or uro-I treated cells lysate processed in the dark. Furthermore, the data revealed that BiP only aggregated as an artifact of light exposure, not in living cells. Lamin A/C and BiP monomers were not detected in the mass spectral analysis because the gel blocks that were cut did not include the region where lamin A/C and BiP monomers migrate. Lastly, collagen type I alpha I chain and fibronectin were less abundant in uro-I treated cells lysate processed in the dark compared to control (data not shown). Interestingly, there was no collagen or fibronectin detected in the light processed cell lysate This confirms that loss of monomer is a reliable read out for protein aggregation and that bone matrix proteins are likely forming high molecular weight aggregates that are unable to migrate into the gel.

FIGS. 8A-8B. Full-length blots/gels. Uncropped blots and gels from FIG. 3C (FIG. 8A) and FIG. 3G (FIG. 8B). Membrane/gel edges are shown. Dashed lines represent non-adjacent lanes in the gel.

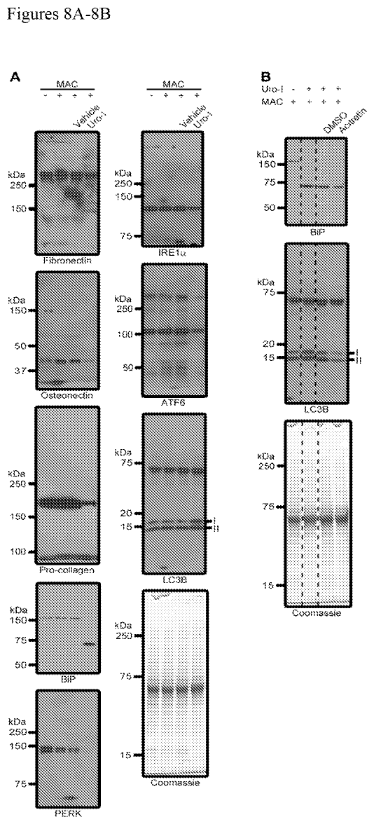

### DETAILED DESCRIPTION

The following definitions are used, unless otherwise described. Alkyl, alkoxy, alkenyl, etc. denote both straight and branched groups, but reference to an individual group such as propyl embraces only the straight chain moiety, a branched chain isomer such as isopropyl being specifically identified.

The term “alkyl”, by itself or as part of another substituent, means, unless otherwise stated, a straight or branched chain hydrocarbon, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkenyl” refers to an unsaturated alkyl group having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers. In a chemical formula. when a double bond is depicted with crossed lines:

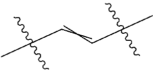 [SMILES: *C=C*]

the formula includes both cis and trans isomers. When a double bond is shown as either cis or trans, it signifies the specific isomer.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “alkoxycarbonyl” as used herein refers to a group (alkyl)-O—C(═O)—, wherein the term alkyl has the meaning defined herein.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological or pathologic change or disorder, such as, for example, the development or spread of tissue damage. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. In addition to mortality, it can also include improved morbidity. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound that (i) treats or prevents the particular disease, condition, or disorder, (ii) attenuates, ameliorates, or eliminates one or more symptoms or manifestations of the particular disease, condition, or disorder, or (iii) prevents or delays the onset of one or more symptoms of the particular disease, condition, or disorder described herein.

The term “animal” includes mammals. The term “mammal” includes humans, higher non-human primates, rodents, domestic, cows, horses, pigs, sheep, dogs and cats. In one embodiment, the mammal is a human.

The term “retinoid” includes compounds that reduce uroporphyrin-I (uro-I) accumulation. For example, the term includes both natural and synthetic analogs of Vitamin A. A specific retinoid is retinol, tretinoin, isotretinoin, alitretinoin, acitretin, adapalene, bexarotine, or tazarotene or a pharmaceutically acceptable salt thereof.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Specific values listed below for substituents, and ranges, are for illustration only; they do not exclude other defined values or other values within defined ranges for the moieties and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, adamantyl, or cyclohexyl; (C₁-C₆)alkoxy can be methoxy, ethoxy, propoxy, isopropoxy, butoxy, iso-butoxy, sec-butoxy, pentoxy, 3-pentoxy, or hexyloxy; and (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl.

A specific retinoid is a compound of formula I:

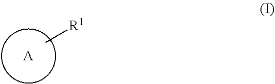

wherein:
ring A is phenyl, cyclopentene-1-yl, or cyclohexen-1-yl, which phenyl, cyclopentene-1-yl, or cyclohexen-1-yl is optionally substituted with one or more groups independently selected from (C₁-C₈)alkyl, (C₃-C₁₀)cycloalkyl, (C₁-C₈)alkoxy, and (C₃-C₈)cycloalkyloxy; and
R¹ is (C₅-C₂₀)alkenyl that is substituted with one or more groups independently selected from hydroxy, carboxy, or (C₁-C₆)alkoxycarbonyl;
   or a pharmaceutically acceptable salt thereof.

A specific ring A is substituted with one or more groups independently selected from (C₁-C₈)alkyl, (C₃-C₁₀)cycloalkyl, (C₁-C₈)alkoxy, and (C₃-C₈)cycloalkyloxy.

A specific ring A is substituted with one or more groups independently selected from (C₁-C₈)alkyl and (C₁-C₈)alkoxy.

A specific ring A is substituted with one or more groups independently selected from (C₁-C₈)alkyl.

A specific ring A is substituted with one or more (C₁-C₈)alkyl and with one or more (C₁-C₈)alkoxy.

A specific compound or pharmaceutically acceptable salt is a compound of formula (Ia):

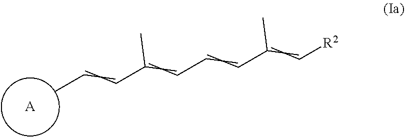

wherein:
R² is hydroxymethyl, carboxy, or (C₁-C₆)alkoxycarbonyl;
   or a pharmaceutically acceptable salt thereof.

A specific compound or pharmaceutically acceptable salt is a compound of formula (Ib):

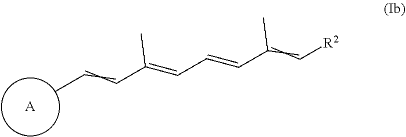

wherein:
R² is hydroxymethyl, carboxy, or (C₁-C₆)alkoxycarbonyl;
   or a pharmaceutically acceptable salt thereof.

A specific compound or pharmaceutically acceptable salt is a compound of formula (Ic):

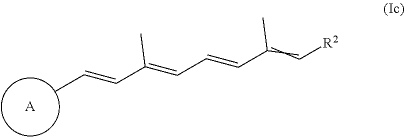

wherein:
R² is hydroxymethyl, carboxy, or (C₁-C₆)alkoxycarbonyl;
   or a pharmaceutically acceptable salt thereof.

A specific compound or pharmaceutically acceptable salt is a compound of formula (Id):

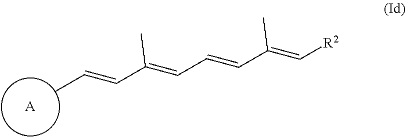

wherein:
R² is hydroxymethyl, carboxy, or (C₁-C₆)alkoxycarbonyl;
   or a pharmaceutically acceptable salt thereof.

A specific compound or pharmaceutically acceptable salt is a compound of formula (Ie):

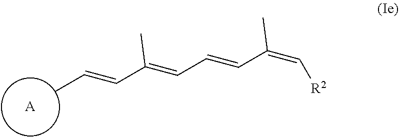

wherein:
R² is hydroxymethyl, carboxy, or (C₁-C₆)alkoxycarbonyl;
   or a pharmaceutically acceptable salt thereof.

A specific ring A is phenyl that is optionally substituted with one or more groups independently selected from (C₁-C₈)alkyl, (C₃-C₁₀)cycloalkyl, (C₁-C₈)alkoxy, and (C₃-C₈)cycloalkyloxy.

A specific ring A is cyclopentene-1-yl that is optionally substituted with one or more groups independently selected from (C₁-C₈)alkyl, (C₃-C₁₀)cycloalkyl, (C₁-C₈)alkoxy, and (C₃-C₈)cycloalkyloxy.

A specific ring A is cyclohexen-1-yl that is optionally substituted with one or more groups independently selected from (C₁-C₈)alkyl, (C₃-C₁₀)cycloalkyl, (C₁-C₈)alkoxy, and (C₃-C₈)cycloalkyloxy.

A specific ring A is selected from the group consisting of:

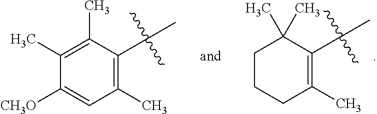

A specific compound or pharmaceutically acceptable salt is selected from the group consisting of:

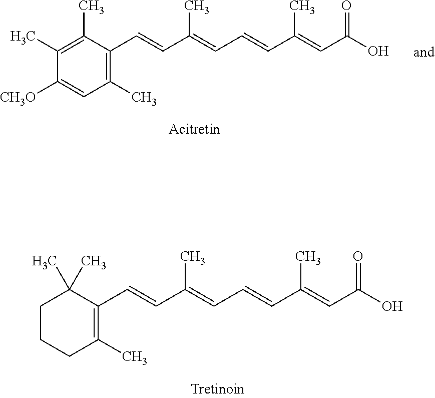

and pharmaceutically acceptable salts thereof.

Administration of a retinoid as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The retinoids can be formulated as pharmaceutical compositions and administered to a mammalian host, such as a human in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the animal's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant or salt. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver a retinoid to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of a retinoid can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the animal, and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Methods of the Invention

The invention also provides a method for identifying test compounds that are useful for reducing porphyrin binding in bones or for treating conditions associated with porphyrin accumulation, such as, for example, congenital erythropoietic porphyria. The method comprises:
injecting a porphyrin (e.g. Uro-I) into a zebrafish;
contacting the zebrafish with a target compound in a medium;
measuring the accumulation of the porphyrin in the bones or other tissue of the zebrafish;
comparing the accumulation of the porphyrin in the bones or other tissue of the zebrafish with a control to determine whether the target compound reduced porphyrin accumulation in the bones or other tissue of the zebrafish; and
optionally determining the amount of porphyrin in the medium.

In one embodiment, the medium comprises water. In another embodiment, the medium comprises water and nutrients. In one embodiment, the accumulation of Uro-I in the bones of the zebrafish is measured about 12-48 hours after contacting with the target compound. In one embodiment, the accumulation of Uro-I in the bones of the zebrafish is measured about 24 hours after contacting with the target compound.

Techniques that can be used to perform the methods of the invention are described by Elenbaas, J. S., et al., FASEB, 2016, 30, 1798-1810; and Cunha, J. B., Scientific Reports, 2021, 11, 9601. For example, the method can be carried out as follows: Six dpf AB×TL zebrafish larvae injected with uro-I were immediately transferred to 10 cm plastic dishes containing 10 μM acitretin (test compound) or DMSO (control) in E3 medium, and incubated for 24 hours in the dark at 28.5° C.; Porphyrin binding to bones and bone volume were analyzed in the acitretin treated and control zebrafish by confocal microscopy using an Olympus FV500 confocal microscope (10× objective, confocal aperture of 300 micron) with an optical thickness of 10 μm and z-step size of 10 μm.

The invention will now be illustrated by the following non-limiting Examples.

### EXAMPLE

### Example 1

### Introduction

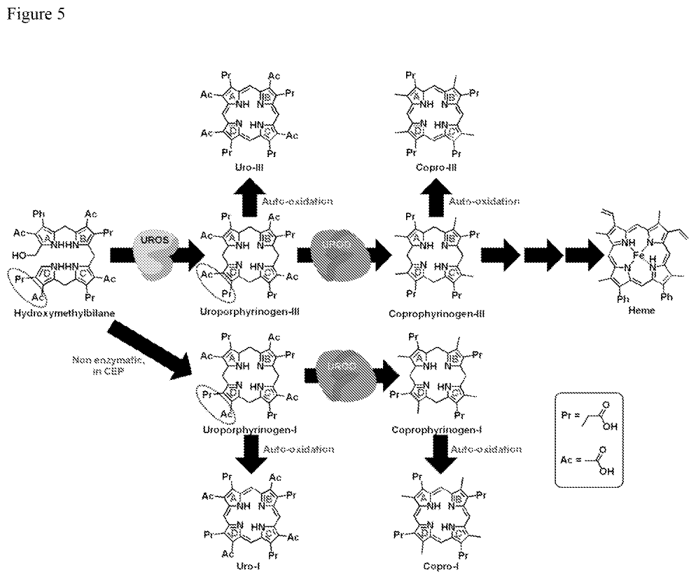

Porphyrias are a group of inherited disorders due to defects in the heme biosynthetic pathway (Puy, H., et al., The Lancet 375, 924-937, doi:10.1016/S0140-6736(09)61925-5 (2010); and Ajioka, R. et al., Biochimica et Biophysica Acta (BBA)—Molecular Cell Research 1763, 723-736, doi:https://doi.org/10.1016/j.bbamcr.2006.05.005 (2006)). One such example is congenital erythropoietic porphyria (CEP), most commonly caused by loss of function mutation in uroporphyrinogen III synthase (FIG. 5), the third step of the heme biosynthetic pathway. CEP is rare, with ˜250 cases reported to date. It is autosomal recessive and associated with reduced UROS activity (5% of normal) and consequent accumulation of uro/coproporphyrin-I (uro/copro-I) in bone marrow, erythrocytes, plasma, and increased uro/copro-I excretion in urine and stool. CEP is characterized by severe photosensitivity, with skin fragility and blistering of sun-exposed areas. Scaring due to secondary skin infections and bone resorption contribute to disfigurement of light-exposed areas. Other clinical manifestations of this multisystem disease include chronic ulcerative keratitis, hemolysis, which may require repeated blood transfusions in severe cases, nonimmune hydrops fetalis, red urine since birth, erythrodontia and osteodystrophy. Currently, there is no specific pharmacological treatment for CEP, with interventions being life-style-related (e.g. avoidance of sun) or complex procedures, including bone marrow transplantation.

Fluorescent porphyrin accumulation in porphyria causes organelle specific protein-oxidation and aggregation through mechanisms that involve type-II photosensitive reactions and secondary oxidative stress. Porphyrin-mediated protein aggregation in CEP potentially plays a major mechanistic role in tissue damage that involves accumulation of fluorescent uro/copro-I.

Uro-I injection of zebrafish larvae was found to mimic features of CEP, including uro-I accumulation in bones and bone deformation, as judged by decreased vertebra and operculum volume. Uro-I treatment of an osteoblastic human osteosarcoma cell line, Saos-2, caused significant decrease in mineral matrix synthesis and proteotoxicity. Using high-throughput drug screening, acitretin, a 2^{nd} generation retinoid, was identified as an effective drug that mitigates some of the harmful effects of uro-I in zebrafish and Saos-2 cells.

### Results

### An Inducible Zebrafish Model Mimics Bone Defects of Human CEP

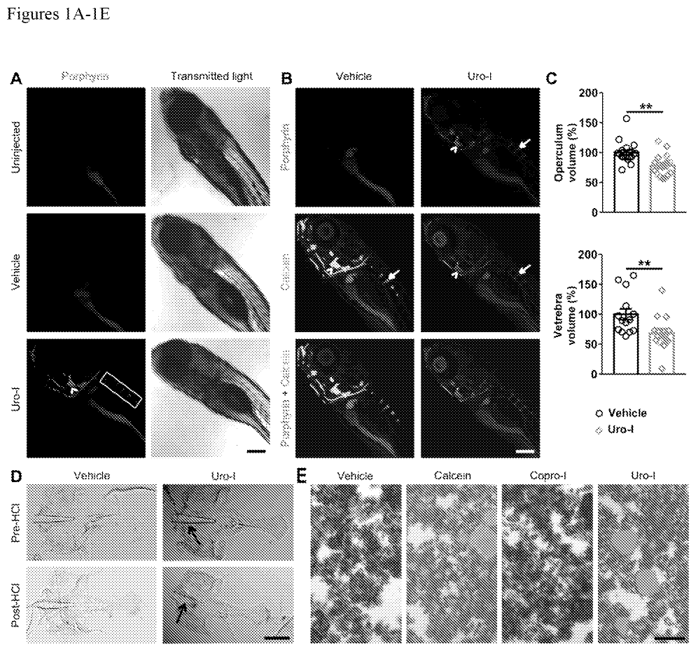

Uro-I injected zebrafish larvae showed porphyrin fluorescence in bone tissue (FIG. 1A). To confirm that uro-I binds specifically to bone, larvae were co-injected with calcein (bone-specific dye) and imaged. Calcein and uro-I fluorescence co-localized (FIG. 1B), confirming uro-I bound to bone. Additionally, uro-I-injected larvae exhibited severe photosensitivity and had to be shielded from light to prevent their death. Next uro-I-mediated bone defect was assessed by measuring the volume of the operculum and 4^{th} vertebra. Notably, uro-I injection significantly decreased operculum and 4^{th} vertebra volume (FIG. 1C).

Bone matrix is composed of protein/organic (including collagen/fibronectin/osteonectin) and inorganic components (minerals, mostly hydroxyapatite). Whether uro-I binds to the protein/organic or inorganic parts of bone matrix was investigated by demineralizing bones of uro-I-injected larvae. Demineralization caused loss of uro-I fluorescence, indicating that uro-I is extractable from the mineral matrix (FIG. 1D). This finding was validates in vitro using hydroxyapatite crystals. Uro-I, but not copro-I, bound to hydroxyapatite, with calcein binding used as a positive control (FIG. 1E). Therefore, uro-I binds to the inorganic bone matrix and its administration to zebrafish phenocopies three major features of CEP: osteal accumulation, bone defects, and severe photosensitivity.

### Acitretin Mitigates Uro-I Effects in Bones of Zebrafish Larvae

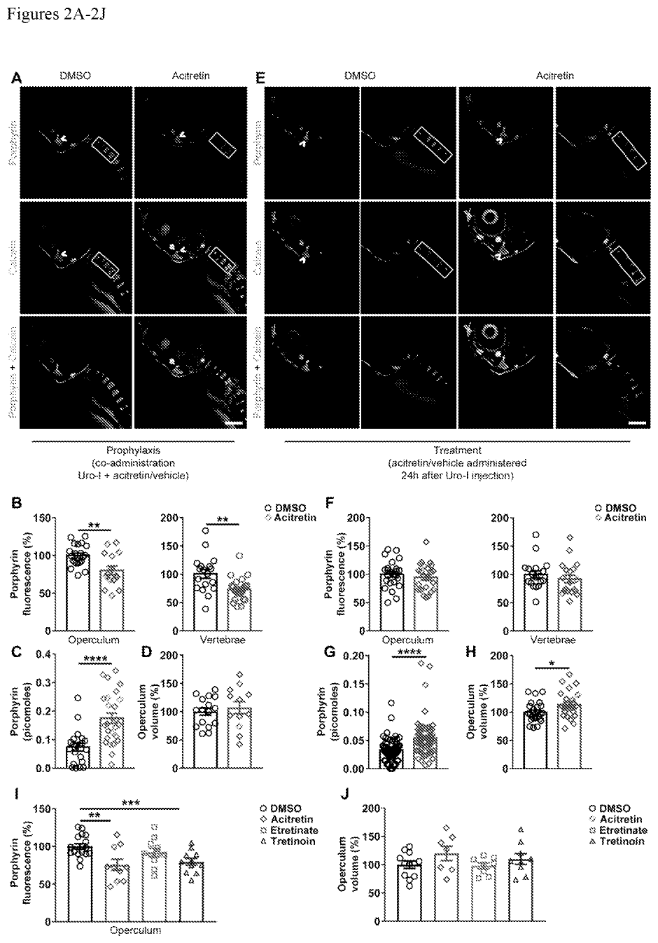

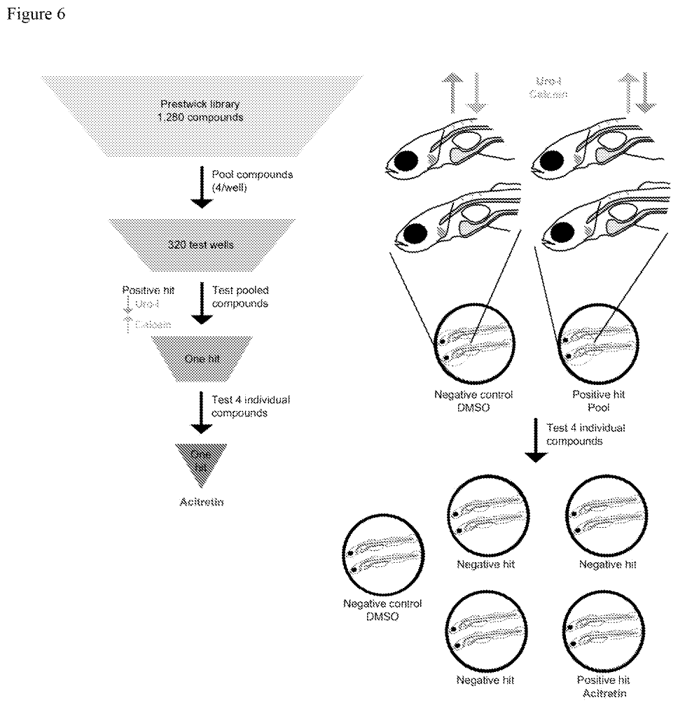

To identify potential drugs to treat CEP, high-throughput screening of 1,280 small molecules was carried out by co-administering drug and uro-I injection in the zebrafish CEP model (FIG. 6). Acitretin, a second-generation retinoid commonly used to treat psoriasis, decreased uro-I accumulation in bones. The screening results were validated and acitretin was further characterized as a potential treatment for CEP by testing whether it had a prophylactic effect. Uro-I injected larvae were immediately transferred to either acitretin- or vehicle-containing medium. After 24 hours, acitretin-treated larvae had significantly reduced porphyrin fluorescence in their bones (operculum and vertebrae FIGS. 2A and 2B) and increased uro-I excretion into the medium, but there was no effect on operculum volume (FIG. 2D). To assess the therapeutic potential of acitretin, larvae were injected with uro-I then transferred after 24 hours to medium containing either acitretin or vehicle and incubated for further 24 hours then imaged. Acitretin did not decrease bone porphyrin fluorescence (FIGS. 2E and 2F), but it increased uro-I excretion to the medium and operculum volume (FIGS. 2G and 2H).

Since acitretin is a retinoid, the protective effects of other retinoids were evaluated. Etretinate (second-generation retinoid and precursor of acitretin), tretinoin (all trans-retinoic acid) or acitretin were co-administered to zebrafish with uro-I. In addition to acitretin, tretinoin significantly reduced bone porphyrin (FIG. 2I), but did not prevent loss in bone volume (FIG. 2J). Thus, both retinoids, acitretin and tretinoin, prevent uro-I accumulation in zebrafish larvae. Hence, acitretin attenuated uro-I-mediated bone damage by modulating the dynamics of uro-I bone binding and excretion. Under the conditions tested, etretinate did not demonstrate significant activity.

### Uro-I Impairs Osteoblastic Mineralization by Aggregating Matrix Proteins, Promoting ER Stress and Inhibiting Autophagy

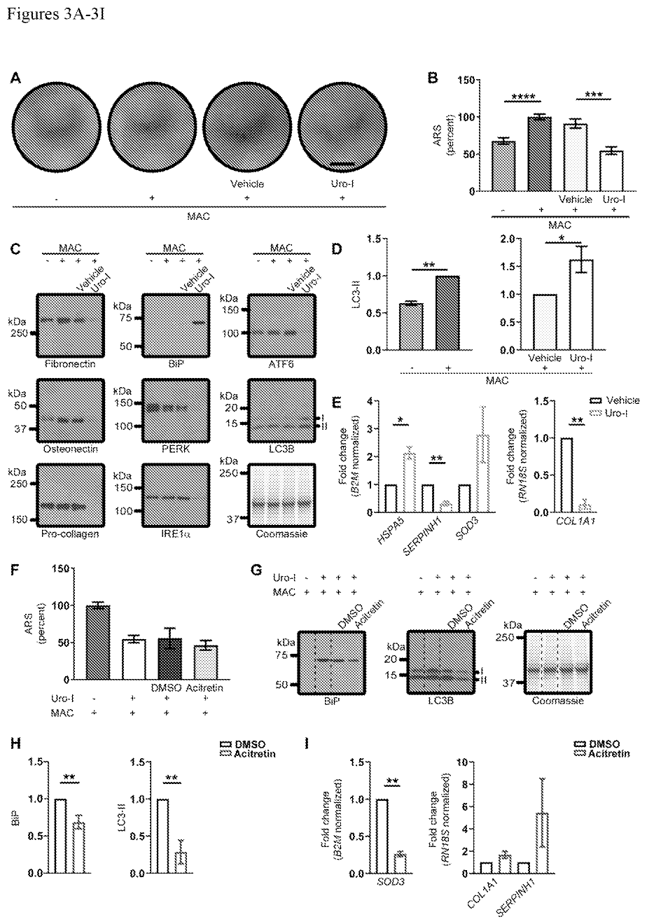

Saos-2 cells, a human osteosarcoma cell line with osteoblastic features, were used to elucidate the molecular mechanism of uro-I-mediated bone damage. Mineralization was stimulated by treating cells with a mineralization activation cocktail (MAC) and measuring alizarin red S (ARS) staining. Saos-2 cells manifested a mineralization phenotype when cultured for 3 days in MAC-supplemented medium, while in uro-I+MAC supplemented medium, mineralization decreased significantly (FIGS. 3A and 3B). Uro-I also caused marked photosensitivity, leading to cell death when cells were not shielded from light.

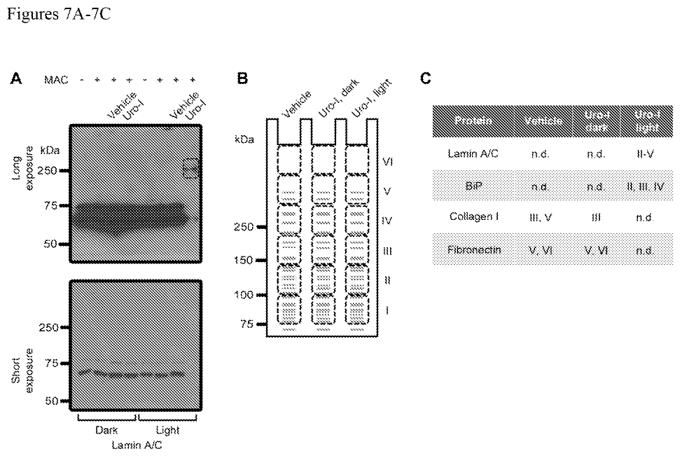

Since fluorescent porphyrins cause protein aggregation or loss of antibody reactivity when tested by immunoblotting, whether uro-I-mediated inhibition of Saos-2 mineralization led to aggregation of bone matrix proteins was tested. Blotting Saos-2 cell lysates prepared from uro-I or vehicle treated cells using antibodies to fibronectin, osteonectin and type 1 pro-collagen showed a distinct loss of monomer for these proteins after uro-I treatment (FIG. 3C). The loss of antibody reactivity was attributed to epitope masking after uro-I binding and subsequent oxidation and aggregation, as shown previously for PP-IX (Maitra, D. et al. Stress. J Biol Chem 290, 23711-23724, doi:10.1074/jbc.M114.636001 (2015)). The loss of matrix protein monomers and aggregation was verified by mass spectrometry (FIG. 7).

Given the effect of uro-I on protein aggregation, whether uro-I treatment initiates unfolded protein response (UPR) and endoplasmic reticulum (ER) stress was tested. Upregulation of BiP, consistent with UPR and ER stress was found (FIG. 3C). Other ER stress markers, including PERK, IRE1α and ATF6, were likely oxidized and aggregated, as judged by monomer loss (FIG. 3C). These findings suggest a non-canonical form of ER stress, which has been observed upon PP-IX accumulation (Maitra, D. et al. Stress. J Biol Chem 290, 23711-23724, doi:10.1074/jbc.M114.636001 (2015)), that involves aggregation and possibly inactivation of ER resident proteins and chaperones.

Autophagy modulates exocytosis of hydroxyapatite crystals and thus plays an important role in bone mineralization by osteoblasts (Nollet, M. et al. Autophagy 10, 1965-1977, doi:10.4161/auto.36182 (2014); and Yin, X. et al. Biochim Biophys Acta Gen Subj 1862, 532-546, doi:10.1016/j.bbagen.2017.11.005 (2018)). Since uro-I inhibited mineralization, whether it also disrupted autophagy was also tested. MAC-treated Saos-2 cells showed increased LC3-II (FIG. 3D, left panel). Uro-I treatment also increased LC3-II levels (FIG. 3D, right panel). The likely explanation for the increased LC3-II is not increased autophagy but a slowing of autophagic flux, which could lead to stalling of exocytosis of mineral-loaded vesicles and decreased mineralization of bone matrix.

To further characterize uro-I-mediated impairment of mineralization in Saos-2 cells, gene expression analysis was performed to probe for alterations in the stress response pathway. Genes that were differentially regulated two-fold or more after uro-I treatment were assessed further. Uro-I treatment increased HSPA5 (2.1×) and SOD3 (2.8×), while SERPINH1 decreased (3.3×) (FIG. 3E, left panel). Since HSPA5 encodes BiP, HSPA5 upregulation supports the BiP upregulation observed biochemically (FIG. 3C). SERPINH1, a collagen-specific chaperone, downregulation may account for collagen misfolding and aggregation. Because type 1 collagen is the most abundant protein in bone matrix, COL1A1 expression was assessed. A 90% reduction in COL1A1 after uro-I treatment was observed (FIG. 3E, right panel). This finding supports uro-I-induced loss of mineralization, since collagen serves as a matrix for mineral deposition.

Whether acitretin can protect from the effects of uro-I was assessed, by treating Saos-2 cells with uro-I in the presence of acitretin. Although acitretin did not prevent uro-I-mediated loss of mineralization (FIG. 3F); it blunted the ER stress response by reducing BiP level and normalized the autophagic flux by reducing LC3-II (FIGS. 3G and 3H). Acitretin also downregulated SOD3 3.8-fold, thereby suggesting that acitretin mitigates the oxidative stress caused by uro-I (FIG. 3I). Upregulation of COL1A1 (1.7×) and SERPINH1 (5.4×) was also observed.

Taken together, this data demonstrates that acitretin mitigates the cellular proteotoxicity and oxidative stress that is caused by uro-I. However, under the conditions tested, acitretin did not rescue the mineralization phenotype caused by uro-I treatment of Saos-2 cells. A possible explanation for why mineralization was not normalized by acitretin is that ER stress and autophagy pathways need to be normalized in order for cells to have their mineralization ability restored. Alternatively, acitretin may act differently on various cell types which is one major advantage offered by the in vivo zebrafish system.

### Discussion

Uro-I is a fluorescent porphyrin capable of types I/II-photosensitized reactions, which explains the observed photosensitivity in CEP, damage to digits and facial features. However, light is unlikely to reach deep internal tissues, which are also affected in CEP. Of note, uro-I-mediated protein aggregation and decreased mineralization was observed in the dark. Previous studies had also reported dark effects of porphyrins. For example, uro-I increased collagen biosynthesis in human skin fibroblasts (Varigos, G., et al., J Clin Invest 69, 129-135, doi:10.1172/jci110423 (1982)), and inhibited erythrocytic uroporphyrinogen decarboxylase activity (Afonso, S. G., et al., Journal of Enzyme Inhibition 5, 225-233, doi:10.3109/14756369109080061 (1991)). A 2-hit model could explain light-independent porphyrin-mediated protein aggregation and proteotoxicity whereby, in absence of light, a secondary oxidant source (e.g., inflammatory cells) causes protein oxidation followed by porphyrin binding to oxidized protein, yielding protein aggregates (Maitra, D., et al., Cell Mol Gastroenterol Hepatol 8, 535-548, doi.org/10.1016/j.jcmgh.2019.06.006 (2019)). CEP is frequently associated with superinfections and osteolysis. Hence, infiltrating immune cell-generated oxidants might serve as a secondary source of oxidant, leading to uro-I mediated protein aggregation in internal organs such as bones. Additionally, uro-I might generate oxidants by acting as a substrate for ferredoxin/ferredoxin:NADP+ oxidoreductase system (Morehouse, K. M., et al., Arch Biochem Biophy 283, 306-310, doi: 10.1016/0003-9861(90)90647-h. (1990). Although ferredoxin/ferredoxin:NADP+ oxidoreductase are commonly associated with hepatic microsomes, they are also expressed in bone and could metabolize uro-I to generate oxidants in the absence of light.

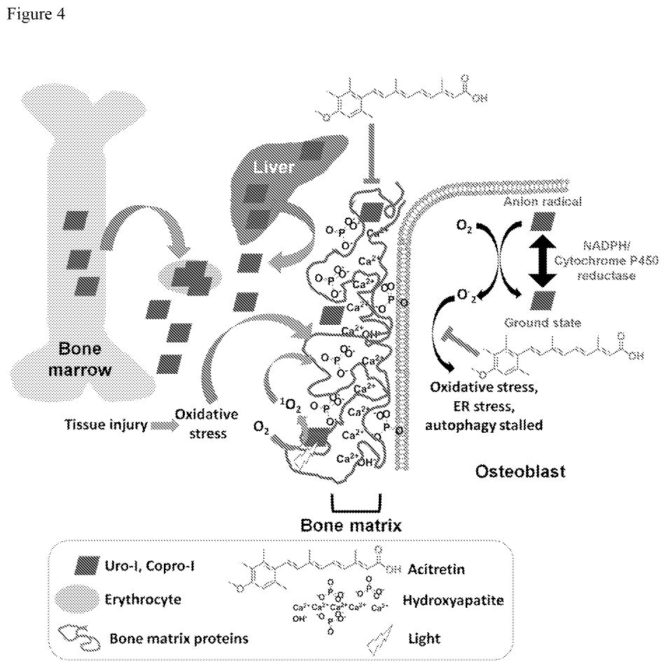

The differences in charge and polarity of uro-I and PP-IX might explain the striking difference in their tissue localization. Retro-orbitally injected PP-IX accumulated in zebrafish liver, while uro-I accumulated preferentially in bone (FIG. 1). Of note, liver cancer cell lines do not uptake uroporphyrin, possibly due to its high negative charge that prevents traversing the cell membrane. Based on our data, we propose that negatively charged uro-I binds to Ca²⁺ in hydroxyapatite (FIG. 4) and thus bone and Saos-2 cells are affected by uro-I. This association with bone matrix causes uro-I to have a different protein aggregation signature compared to PP-IX, which is primarily internalized. PP-IX aggregated intracellular proteins such as keratins and glyceraldehyde 3-phosphate dehydrogenase, whereas uro-I affected extracellular bone matrix proteins (FIG. 3). Oxidants such as singlet oxygen, a major oxidant produced by photosensitive reactions, have extremely small intracellular diffusion distance (10-20 nm) and lifetime (10-40 ns). Binding of uro-I to bone matrix causes a ‘sensitizer-acceptor’ coupling, as observed for other diffusible oxidants, and greatly increases the oxidation efficiency and specificity. Of note, oxidized fibronectin reduces mineralization of rat calvarial osteoblasts in vitro. The high selectivity of uro-I localization to bone matrix might provide a pathway to develop photodynamic therapeutic agents for bone cancers such as osteosarcoma.

The management of CEP is challenging, with current therapeutic options focusing on bone marrow/hematopoietic stem cell transplantation, and by avoidance of sun and light exposure, including the use of protective clothing. There are also potential experimental therapeutic approaches including gene therapy, proteasomal inhibitors, iron chelation, and phlebotomies. Most recently, the repurposed use of ciclopirox, an approved antifungal, showed promising results in the treatment of CEP using a mouse mode (Urquiza, P. et al., Science translational medicine 10, doi:10.1126/scitranslmed.aat7467 (2018). However, there are limitations to these approaches, such as complications from transplantation and neurotoxic side effects of proteasome inhibitors. Currently there are no known pharmaceuticals that act by clearance of uro-I, and in this regard acitretin provides a novel approach. Acitretin might also act as an antioxidant (FIG. 4) due to its hyperconjugated nucleophilic double bonds. Thus, through a combination of destabilizing uro-I-bone matrix interaction and antioxidant activity, acitretin could ameliorate CEP manifestations (FIG. 4).

### Materials and Methods

### Zebrafish Experiments and Cell Culture

Zebrafish (Danio rerio) experiments were conducted using AB×TL hybrid and NHGRI-1 wild type zebrafish lines. All animal procedures were approved by the Rutgers University Institutional Animal Care and Use Committee (protocol number PROTO201900147) and performed in compliance with federal guidelines and the standards of the NIH Guide for the Care and Use of Laboratory Animals, the Rutgers University IACUC Policy Handbook and the Animal Research: Reporting of In Vivo Experiments (ARRIVE) guidelines.

Saos-2 cells were purchased from ATCC. Cells were maintained in McCoy 5A medium supplemented with 15% FBS, penicillin/streptomycin, non-essential amino acids, Hepes and L-glutamine. To induce mineralization, cells were treated with mineralization activation cocktail (MAC), consisting of 5 mM b-glycerophosphate, 50 mM ascorbic acid and 10 nM dexamethasone. Uro-I solution preparation and treatment of zebrafish larvae and Saos-2 cells

Uro-I (uroporphyrin-I dihydrochloride; Frontier Scientific, Catalog #: U830-1) was initially resuspended in 0.1M NaOH and the pH was adjusted to neutral using 0.2 M Na₂HPO₄. Six days post fertilization (dpf), ABTL zebrafish larvae were injected via the retro-orbital route with approximately 3 nL of 7.2 mM Uro-I solution and control larvae were injected with vehicle (0.1M NaOH in 0.2M Na₂HPO₄). After injection, larvae were immediately transferred to Petri dishes wrapped with heavy duty aluminum foil and kept in a dark incubator, at 28.5° C., for 24 h. Where indicated, 7 dpf larvae were injected with approximately 2 nL of 0.2% w/v calcein (Sigma, Catalog #: C0875) 2 h prior to imaging.

Saos-2 cells were plated in 12-well plates (1.5×10⁵ cells/well) and allowed to attach overnight. Cells were then treated with Uro-I (144 mM final concentration) or vehicle in medium containing MAC for 3 days. Experiments were conducted in a dark room and cells were kept shielded from light in a tissue culture incubator.

### Confocal Microscopy Imaging and Quantification

Seven dpf AB×TL zebrafish larvae were anesthetized with tricaine-S (Syndel) and immobilized in 0.5% low melt agarose. Fluorescent z-series were captured using an Olympus FV500 confocal microscope (10× objective, confocal aperture of 300 mm) with an optical thickness of 10 mm and z-step size of 10 mm. Calcein was excited with a 488 nm argon laser and emission was captured between 505 and 525 nm. Porphyrin was excited with a 405 nm laser diode and emission was captured above 560 nm. Three-dimensional image reconstruction and quantification of fluorescent signal and bone volume were performed using Imaris 3D visualization and analysis software v7.7 (Bitplane).

### In Vivo and In Vitro Binding of Uro-I

Six dpf AB×TL zebrafish larvae were injected with Uro-I or vehicle (as described above) and 24 hours later were euthanized by tricaine-S overdose on ice bath. Bone harvest was conducted as previously described (Kessels, M. Y. et al. PLoS One 9, e90568, doi:10.1371/journal.pone.0090568 (2014)). Briefly, soft tissue was removed by incubating larvae with Accumax solution (MilliporeSigma, Catalog #: A7089) under vigorous shaking. Bones were collected using a 70 μm cell strainer, followed by demineralization with 1.2M HCl. Fluorescent images were captured prior to and after the demineralization step using a Zeiss Axio Imager M2 fluorescence microscope. Porphyrin signal was captured using the red fluorescent channel. 10 mg Hydroxyapatite (Acros Organics, Catalog #: 1306-06-5) was incubated with 1 mM Uro-I, 1 mM Copro-I (coproporphyrin-I dihydrochloride, Frontier Scientific, Catalog #: C654-1), vehicle or 0.2% calcein for 30 min in the dark and vortexed every five minutes. Samples were washed and imaged by epifluorescence microscopy as described above.

### High Throughput Drug Screening

Unbiased high throughput drug screening was performed using the Prestwick library (Prestwick Chemical), which consists of 1,280 small molecules chosen by the manufacturer for their bioavailability and safety. A pooled approach, where four compounds were tested together, was used in order to optimize animal use and investigation of drugs with potential for CEP treatment. Zebrafish E3 medium (100 mL/well) was transferred to a 96-well half area imaging plate (Corning, cat. n. 3880) using a Multidrop dispenser (ThermoFisher Scientific). Compounds (0.4 mL of 2 mM stock) were added to the wells using a multichannel plate handling robot (Biomek FX, Beckman Coulter Life Sciences). This step was performed four times in order to pool four compounds into one well: one 384-well stock plate yielded one 96-well test plate. Control wells contained 1.6 mL of DMSO.

Six dpf NHGRI-1 zebrafish larvae were injected retro-orbitally with approximately 2 nL of a solution of Uro-I (10 mM) and calcein (0.2% w/v). Immediately after injection, larvae were transferred to a 96-well test plate (two larvae in 50 mL of E3 medium/well), including the DMSO control wells. Control larvae injected with the drug vehicle (dimethyl sulfoxide, DMSO) and calcein were transferred to E3 medium-only containing wells. Larvae were kept in the dark, at 28.5° C. After 24 h, they were anesthetized with tricaine-S, centrifuged at 500×g for two minutes and imaged using the ImageXpress Micro Cellular Imaging and Analysis System (Molecular Devices). Positive hits were selected based on visual identification of calcein signal increase and porphyrin signal decrease compared to DMSO-treated larvae. Compounds in test wells that met the inclusion criterion were tested individually in the same manner as described above (FIG. 6).

### Acitretin Validation and Treatment

A dose-response curve with acitretin (Selleck Chemicals, Houston, TX) was conducted (0.5-12.5 mM) and 10 mM was observed to yield consistent results, without being toxic to zebrafish larvae. Validation and characterization of acitretin as a potential treatment for CEP was performed. Six dpf ABTL zebrafish larvae injected with uro-I were immediately transferred to 10 cm plastic dishes containing 10 μM acitretin or DMSO in E3 medium (prophylaxis protocol FIG. 2A) and incubated for 24 hours in the dark at 28.5° C. Porphyrin binding to bones and bone volume were analyzed by confocal microscopy as described above. Porphyrin excretion into the medium was quantified. Uro-I-injected larvae were transferred to 96-well plates, one larva/well, 100 mL of 10 mM acitretin or DMSO/well. Medium was collected after 24 hours and porphyrin was quantified as described previously.

Etretinate (Selleck Chemicals, Houston, TX) and tretinoin (Selleck Chemicals, Houston, TX) treatment was performed as described for acitretin. In addition to being used as prophylaxis, the therapeutic effect of acitretin was evaluated. Six dpf AB×TL zebrafish larvae were injected with Uro-I and 24 hours later they were transferred to E3 medium containing 10 mM acitretin or DMSO. Porphyrin binding, excretion and bone volume were analyzed. Saos-2 cells were treated with 10 mM acitretin or DMSO in medium containing MAC and Uro-I.

### Alizarin Red S (ARS) Staining and Quantification

Cell mineralization was quantified by ARS (Sigma Aldrich St. Louis, MO) staining as described previously (Harper, E. et al. PLoS One 12, e0188192, doi:10.1371/journal.pone.0188192 (2017)), with minor modifications. Briefly cells were fixed with 100% ethanol at 37° C. for 1 hour, stained with 40 mM (pH 4.2) ARS solution for 20 minutes in an orbital shaker. Cells were washed and ARS was extracted by incubation of fixed cells with 10% (v/v) acetic acid, followed by scraping, incubation of suspension (85° C., 10 minutes), centrifugation and neutralization of supernatant with 10% (v/v) ammonium hydroxide. ARS standard curve (from 2-0.02 mM) and samples were transferred in triplicate to a 96-well plate and absorbance was measured at 405 nm.

### Cell Harvest, Immunoblotting and Mass Spectrometry

Saos-2 cells were lysed in ice cold RIPA buffer (Sigma Aldrich, St. Louis, MO) with protease inhibitor cocktail (Thermo Scientific, Waltham, MA) and scraped. Whole cell lysate was kept in the dark until reducing SDS-PAGE sample buffer was added. Immunoblotting, band densitometry and mass spectrometry were conducted as described previously (Maitra, D. et al. Cell Mol Gastroenterol Hepatol 8, 659-682 e651, doi:10.1016/j.jcmgh.2019.05.010 (2019); and Maitra, D. et al. J Biol Chem 290, 23711-23724, doi:10.1074/jbc.M114.636001 (2015)). Antibodies to the indicated antigens (and sources) are: ATF6, BiP, LC3B (Cell Signaling Technology, Danvers, MA); fibronectin HFN 7.1, pro-collagen SP1.D8, osteonectin AON-1 (Developmental Studies Hybridoma Bank; Iowa City, Iowa); IRE1a, PERK (Invitrogen, Carlsbad, CA); lamin A/C (Santa Cruz Biotechnology, Dallas, TX).

### Gene Expression Profiling

Saos-2 cells RNA was extracted using RNeasy mini kit (Qiagen, Catalog #: 74104). and gene expression was carried using the RT² Profiler PCR Array for human cellular stress responses (Qiagen, Catalog #: PAHS-019ZA) following manufacturer's instructions. A previously described qPCR (Elenbaas, J. S. et al. Gastroenterology 154, 1625-1629 e1628, doi:10.1053/j.gastro.2018.01.024 (2018)) was performed for COL1A1 and SERPINH1 (IDT Integrated DNA Technologies, PrimeTime assay ID Hs.PT.58.15517795 and Hs.PT.56a.26865778, respectively).

### Statistical Analysis

Statistical analysis was performed using GraphPad Prism v8 (GraphPad Software). Unpaired two-tailed Student's t-test was used to determine statistical significance. Error bars represent standard error of the mean. *p<0.05, **p<0.01, ***p<0.001, ****p<0.0001.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method for treating congenital erythropoietic porphyria in an animal, comprising administering a retinoid to the animal.

2. The method of claim 1, wherein the retinoid is a compound of formula I: 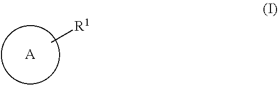
wherein:
ring A is phenyl, cyclopentene-1-yl, or cyclohexen-1-yl, which phenyl, cyclopentene-1-yl, or cyclohexen-1-yl is optionally substituted with one or more groups independently selected from (C₁-C₈)alkyl, (C₃-C₁₀)cycloalkyl, (C₁-C₈)alkoxy, and (C₃-C₈)cycloalkyloxy; and
R¹ is (C₅-C₂₀)alkenyl that is substituted with one or more groups independently selected from hydroxy, carboxy, or (C₁-C₆)alkoxycarbonyl;
or a pharmaceutically acceptable salt thereof.

3. The method of claim 2, wherein the compound or the pharmaceutically acceptable salt is a compound of formula (Ia): 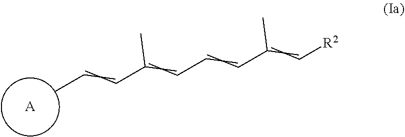
wherein:
R² is hydroxymethyl, carboxy, or (C₁-C₆)alkoxycarbonyl;
or a pharmaceutically acceptable salt thereof.

4. The method of claim 2, wherein the compound or the pharmaceutically acceptable salt is a compound of formula (Ib): 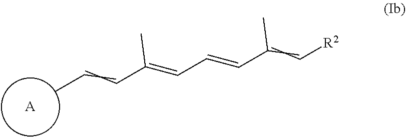
wherein:
R² is hydroxymethyl, carboxy, or (C₁-C₆)alkoxycarbonyl;
or a pharmaceutically acceptable salt thereof.

5. The method of claim 2, wherein the compound or the pharmaceutically acceptable salt is a compound of formula (Ic): 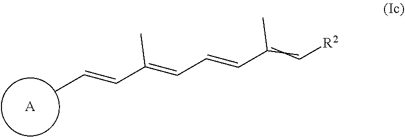
wherein:
R² is hydroxymethyl, carboxy, or (C₁-C₆)alkoxycarbonyl;
or a pharmaceutically acceptable salt thereof.

6. The method of claim 2, wherein the compound or the pharmaceutically acceptable salt is a compound of formula (Id): 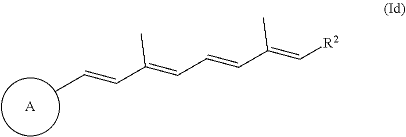
wherein:
R² is hydroxymethyl, carboxy, or (C₁-C₆)alkoxycarbonyl;
or a pharmaceutically acceptable salt thereof.

7. The method of claim 2, wherein the compound or the pharmaceutically acceptable salt is a compound of formula (Ie): 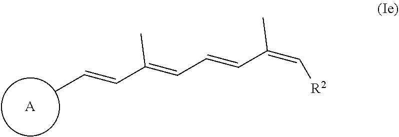
wherein:
R² is hydroxymethyl, carboxy, or (C₁-C₆)alkoxycarbonyl;
or a pharmaceutically acceptable salt thereof.

8. The method of any one of claims 2-7, wherein ring A is phenyl that is optionally substituted with one or more groups independently selected from (C₁-C₈)alkyl, (C₃-C₁₀)cycloalkyl, (C₁-C₈)alkoxy, and (C₃-C₈)cycloalkyloxy.

9. The method of any one of claims 2-7, wherein ring A is cyclopentene-1-yl that is optionally substituted with one or more groups independently selected from (C₁-C₈)alkyl, (C₃-C₁₀)cycloalkyl, (C₁-C₈)alkoxy, and (C₃-C₈)cycloalkyloxy.

10. The method of any one of claims 2-7, wherein ring A is cyclohexen-1-yl that is optionally substituted with one or more groups independently selected from (C₁-C₈)alkyl, (C₃-C₁₀)cycloalkyl, (C₁-C₈)alkoxy, and (C₃-C₈)cycloalkyloxy.

11. The method of any one of claims 2-10, wherein ring A is substituted with one or more groups independently selected from (C₁-C₈)alkyl, (C₃-C₁₀)cycloalkyl, (C₁-C₈)alkoxy, and (C₃-C₈)cycloalkyloxy.

12. The method of any one of claims 2-10, wherein ring A is substituted with one or more groups independently selected from (C₁-C₈)alkyl and (C₁-C₈)alkoxy.

13. The method of any one of claims 2-10, wherein ring A is substituted with one or more groups independently selected from (C₁-C₈)alkyl.

14. The method of any one of claims 2-10, wherein ring A is substituted with one or more (C₁-C₈)alkyl and with one or more (C₁-C₈)alkoxy.

15. The method of any one of claims 2-7, wherein ring A is selected from the group consisting of:

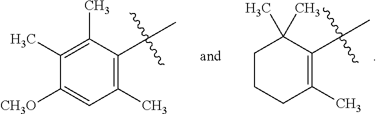

16. The method of claim 2, wherein the compound or the pharmaceutically acceptable salt is selected from the group consisting of: 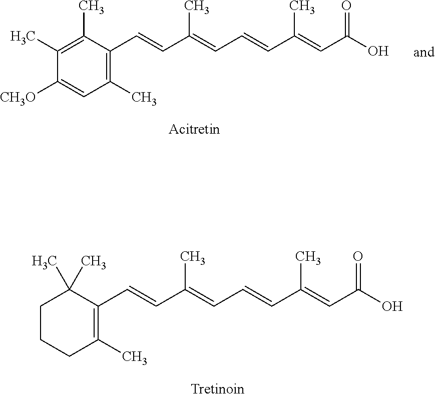
and pharmaceutically acceptable salts thereof.

17. The method of claim 1 wherein the retinoid is retinol, tretinoin, isotretinoin, alitretinoin, acitretin, adapalene, bexarotine, or tazarotene or a pharmaceutically acceptable salt thereof.

18. The method of any one of claims 1-17, wherein the animal is a human.

19. A pharmaceutical composition for treating congenital erythropoietic porphyria comprising a retinoid or a pharmaceutically acceptable salt thereof and a pharmaceutically acceptable excipient.

20. The pharmaceutical composition of claim 19, wherein the retinoid or the pharmaceutically acceptable salt thereof is a compound as described in any one of claims 2-17 or a pharmaceutically acceptable salt thereof.

21. A retinoid for the prophylactic or therapeutic treatment of congenital erythropoietic porphyria.

22. The retinoid of claim 21, that is a compound as described in any one of claims 2-17 or a pharmaceutically acceptable salt thereof.

23. The use of a retinoid to prepare a medicament for treating congenital erythropoietic porphyria.

24. The use of claim 23, wherein the retinoid is a compound as described in any one of claims 2-17 or a pharmaceutically acceptable salt thereof.

25. A method comprising:
injecting a porphyrin into a zebrafish;
contacting the zebrafish with a target compound in a medium;
measuring the accumulation of the porphyrin in the bones or other tissue of the zebrafish;
comparing the accumulation of the porphyrin in the bones or other tissue of the zebrafish with a control to determine whether the target compound reduced porphyrin accumulation in the bones or other tissue of the zebrafish; and
optionally determining the amount of porphyrin in the medium.
